(19) United States
(12) Patent Application Publication
(10) Pub. No.: US 2009/0011521 A1
(43) Pub. Date: Jan. 8, 2009

(54) Streptavidin surface acoustic wave immunosensor apparatus

(75) Inventors: Hsin-Chun Lu, Tao-Yuan (TW); Chung-Yi Wang, Tao-Yuan (TW); Chia-Yen Li, Tao-Yuan (TW); Chung-Wei Chang, Tao-Yuan (TW); Meng-Kai Huang, Tao-Yuang (TW); Hao-Yu Ting, Tao-Yuan (TW); Hsin-Chieh Yang, Tao-Yuan (TW); Yi-Ju Hsiao, Tao-Yuan (TW)

Correspondence Address: BACON & THOMAS, PLLC, 625 SLATERS LANE, FOURTH FLOOR, ALEXANDRIA, VA, 22314-1176, US

(73) Assignee: Chang Gung University, Tao-Yuan (TW)

(21) Appl. No.: 12/213,813

(22) Filed: Jun. 25, 2008

(30) Foreign Application Priority Data
Jun. 25, 2007 (TW) 096122833

(51) Int. Cl. G01N 33/566; H01L 41/22; H01L 41/107

(52) U.S. Cl. 436/501; 29/25.35; 310/313 R

(57) ABSTRACT

The invention, the manufacturing process and operation method for forming the streptavidin surface acoustic wave (SAW) immunosensor apparatus is disclosed. Firstly, the PZT film is formed on silicon substrate by using the micro-powder-sol-gel method. Then, the metal transducer electrodes are coated on the PZT film using semiconductor process technology to produce the SAW. Finally, the sensing area of the SAW element is modified by streptavidin to form the streptavidin SAW immunosensor. The invention could be used for examining the ligand decorated by biotin, also for examining antibody.

### BACKGROUND OF THE INVENTION

1. Field of the Invention

The invention relates to an immunosensor, more particularly to an immunosensor carrying out the inspection using the piezoelectric surface acoustic wave sensing element fixed with streptavidin.

2. Description of the Prior Art

Since the 20th century, the biosensor has already been applied to chemical, biochemical and medical field extensively. It has received the great attention in the field of biotechnology through its high feature, high sensitivity, and simultaneous response ability. Furthermore, the biosensor has combined the interdisciplinary technology such as the biological sensitiveness and the microelectronics and electronic materials etc.

Basically, the biosensor has combined the biological materials, such as the organism tissue, microorganism, organ, cell receptor, enzyme, antibody, nucleic acid etc. The signal of biologically derived material or biomimic intimately material can be transferred by the optical, electrochemical, heat, piezoelectric or magnetic energy through highly sensitive chemical or physical transducer. The signal transferred from the transducer and the output signal can be treated and amplified to become the text and graphic on the analytical devices which is apt to be understood by the user.

In numerous biosensors, the immune biosensor attracts more attention because it has higher sensitivity and specific response than other common sensor.

The piezoelectric crystal is used as the transducer of the immune biosensor in recent years. The earliest piezoelectric crystal is applied in quartz crystal microbalance (QCM). The early piezoelectric crystal sensor is only used for the measurement of air pollutant. Most of cladding materials used on the surface of electrode to get the sensing function are abiotics. Guilbault (1983) was the first person who used the formaldehyde dehydrogenase to fix on the crystal electrode to measure the formaldehyde composition in the air, which had caused the attention of the biochemical sector.

The structure of piezoelectric quartz crystal looks like the sandwich, its quartz substrate is usually sandwiched by two sheets of metal electrode (such as gold, silver, aluminum and nickel etc.). The electrode can introduce an oscillating electric field along the vertical direction of the chip surface. The above-mentioned oscillating electric field forces the crystal lattice to generate a mechanical oscillating behavior similar to the standing wave. When the thickness of the quartz substrate is constant, the mechanical oscillation is expressed by a rated frequency. The resonant frequency can be measured through introducing proper vibration circuit. In the conventional technology, the resonant frequency generated by the coupling of mechanical and electrical oscillation depends on the physical property of quartz chip, such as thickness, density, shear coefficient, and the surface density, viscosity of crystal upon contacting gas or liquid, as well as the pressure difference and temperature between both sheets of chip.

There are many factors which influence the sensing effect of the above-mentioned traditional immunosensor, and cause lower accuracy. The sensing result has the higher error, which needs better immunosensor, especially when the new type piezoelectric element is applied in the immunosensor, it can raise the sensing accuracy.

After the relevant Taiwan patents are inquired, the patent of number 00557139 (Sweden) does not include the technological field of the immunosensor. The patents associated with “immunosensor” include 00476640 (P&G, U.S.A.) and 00421587 (P&G, U.S.A.), which only have basic technology and content of the relevant biological immunosensor.

The US patents associated with “biosensor”, “immunosensor” and “Streptavidin immunosenso” etc. are also inquired. The earliest patent about “Immunosensor” and “Streptavidin” was U.S. Pat. No. 5,156,972 patent issued in 1992. It only has the original basic design of basic immunosensor. U.S. Pat. No. 5,413,939 patent is the combination of immunosensor, wherein the lead zirconium titanate (PZT) is used for some parts of instrument, which does not have surface acoustic wave function. U.S. Pat. No. 6,333,200 patent is the surface immunosensor which can be arranged to a minimization. U.S. Pat. No. 6,670,115 patent is the improvement for the electrode sensing of immunosensor.

### SUMMARY OF THE INVENTION

In accordance with the present invention, a method is provided for forming the streptavidin surface acoustic wave immunosensor apparatus.

The embodiment of the invention is to fix a biomolecular identifying film on the surface of piezoelectric crystal electrode, to detect the corresponding compound, such as the antibody-antigen, enzyme-substrate, hormone-receptor etc. Because the sensitivity of the piezoelectric crystal of this invention can reach a level of 10⁻¹² g, so it is much suitable for the detecting of the biomolecular level.

Firstly, the precursor solution of lead zirconium titanate (PZT) is prepared for the invention. The lead acetate (Pb(OAc)₂. 3H₂O) crystal is ground to powder, and the finished lead acetate is dehydrated. The zirconium, titanium and excess lead are added. After they are dissolved, the vacuum thickening process is used to get the precursor solution with suitable concentration.

The micro-powder-sol-gel process is used to prepare the lead zirconium titanate, which is ground to powder by the ball milling.

The silicon chip substrate coated with electrode is cut to suitable size. It is washed by the fresh water and acetone to remove the oil and grease particles on the substrate.

The spin coating process is used to prepare the lead zirconium titanate film, which is heat treated at different temperature. Put the substrate into a high temperature furnace for the heat treatment.

Upon making the surface acoustic wave sensing element, a layer of gold (conductive metal) electrode is coated on the piezoelectric film to become the Chemical Interaction Material (CIM) layer. The sensing material is then connected to the surface of gold electrode.

The surface of surface acoustic wave sensing element is modified by the streptavidin. The purpose is to fix the detecting antigen on the surface of surface acoustic wave sensing element. The self discharge monomolecular film modification method is used to combine the organism and the piezoelectric transducer. Due to the streptavidin can be easier to be obtained; therefore the streptavidin will be elected for the invention. Not only the streptavidin can be produced by industrialization manufacturing processes, but also the streptavidin will be quite sensitive upon sensing processes.

After the above-mentioned processing, the structure of the streptavidin surface acoustic wave immunosensor apparatus of the invention describes as the followings:

The signal generator, which can offer an external signal; the input converter, which can deal with the input signal; the streptavidin film, which can be used as the bio-probe; the output converter, which can deal with the signal from the bio-probe and the input signal to the input converter; the frequency counter, which can receive the output signal from the output converter; the sensor substrate, which has the lead zirconium titanate layer with piezoelectric property, and the sensor substrate will carry out the surface acoustic wave delivery sensing by using the piezoelectric property.

In addition, the principle of Sauerbrey equation is used for the measurement of the streptavidin surface acoustic wave immunosensor apparatus. The measuring method is described as the followings:

Firstly, connect the sensor and the probe. Secondly, the probe receives the signal generated from the signal generator using the input converter. The operation frequency is inputted into the input converter. The decayed signal is outputted from the output converter. Finally, the signal is converted to the sensing result by the frequency counter. Furthermore, the phase change, frequency change caused by the surface perturbation and the property change of the surface acoustic wave caused by the energy loss of the surface acoustic wave all can be measured to understand the sensitivity and feasibility of the sensor.

The invention can be widely applied to the immunosensor, biosensor, biomedical sensor, biological medicine sensor and biological environment sensor etc for the medical treatment applications.

The invention meets the basic requirement of the immunosensor, including high specificity, high linearity, high signal/noise ratio (S/N ratio), short response time, high reproducibility etc. It can highly meet the requirement of industry.

### BRIEF DESCRIPTION OF THE DRAWINGS

The foregoing aspects and many of the attendant advantages of this invention will become more readily appreciated as the same becomes better understood by reference to the following detailed description, when taken in conjunction with the accompanying drawings, wherein:

FIG. 1 is a flow-chart schematically illustrating the embodiment of the invention;

FIG. 2 is a flow-chart schematically illustrating the embodiment of fixing and modification on the surface of the streptavidin;

FIG. 3 shows the structural drawing for the element for the invention;

FIG. 4 shows the sectional drawing for the element for the invention;

FIG. 5 shows the sensing method for the invention; and

FIG. 6 shows the measurement result for the network analyzer in the invention.

### DESCRIPTION OF THE PREFERRED EMBODIMENT

The following is a description of the present invention. The invention will firstly be described with reference to one exemplary structure. Some variations will then be described as well as advantages of the present invention. A preferred method of fabrication will then be discussed. An alternate, asymmetric embodiment will then be described along with the variations in the process flow to fabricate this embodiment.

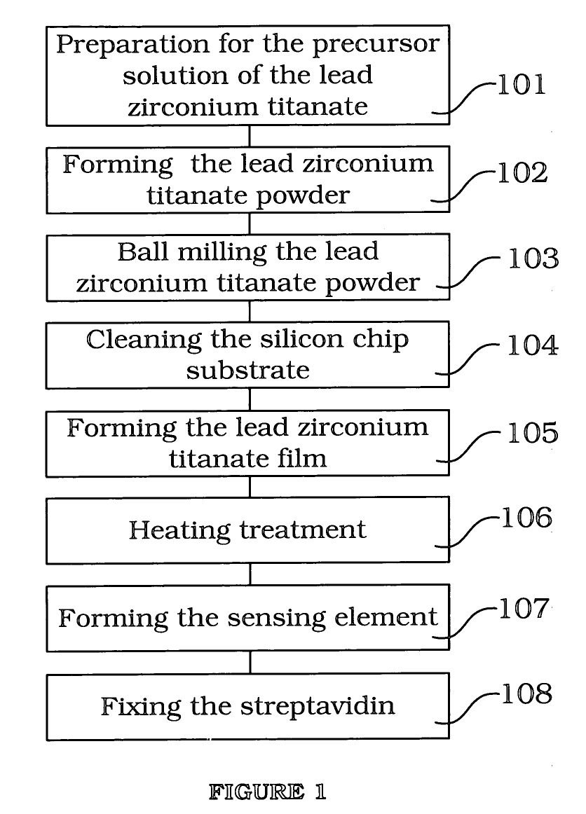

The detailed step of this invention will be described as followings, referring the FIG. 1 as the flow chart of this invention:

The invention uses the powder-sol-gel process to prepare the lead zirconium titanate piezoelectric film, and adopts the semiconductor process technology to coat the inter-digital transducer electrode on the piezoelectric substrate, and then to make the surface acoustic wave sensing element. Then the streptavidin is used to modify the sensing area of element to inspect the adducts with the modified biotins, which can be applied for the inspection of antibody.

The detailed implementation steps of this invention are described as follows:

Firstly, as shown in FIG. 1, designating numeral 101, the precursor solution of the anhydrous lead zirconium titanate (PZT) is prepared. The lead acetate (Pb(OAc)₂ . 3H₂0) crystal is ground to powder-like, and it is heated for 12 hours to dehydrate the lead acetate. The excess lead (10%), reactant and solvent are added in the flask, respectively. After they are dissolved, put three solutions on 0□ ice bath. After staying for 1 hour, mix and agitate three solutions. The vacuum thickening process is used to obtain the anhydrous precursor solution having suitable concentration. Finally, put it in a stationary condition for one day to facilitate the reaction.

As shown in FIG. 1, designating numeral 102, the sol-gel process is used to prepare the anhydrous lead zirconium titanate powder. It means the solvent in the precursor solution is removed to get the sol-gel of lead zirconium titanate. The sol-gel is ground, sintered and sieved to obatin the lead zirconium titanate powder. The so-called micro-powder-sol-gel process is used to dissolve the precursor reactants in water or organic solvent to carry on the hydrolysis and condensation reaction to obtain the sol-gel, and then it is evaporated and dried to become as the sol-gel, then the sol-gel will be heated to become as the crystalline powder.

As shown in FIG. 1, designating numeral 103, processing the ball milling will be carried out for the anhydrous lead zirconium titanate. The sintered lead zirconium titanate powder is ground to micro-powder by wet ball milling method. In order to get the micro-powder with smaller particle size, it can be ground two times. In the first time, the zirconium ball (ZrO) and ethanol is added as the solution, the revolution of wet ball mill is 300 rpm and the grinding time is 15 hours. In order to get smaller particle size and better dispersion, the lead zirconium titanate is used to replace the ethanol as the grinding aid. The precursor solution of lead zirconium titanate, 0.8 mm diameter zirconium ball (ZrO) and different dispersant (Fish oil, Nopcosperse, OL573) are added to the ground micro-powder. After it is ground under 200 rpm for 15 hours, the homogeneous coating solution can be obtained, and filtered with the filter paper.

As shown in FIG. 1, designating numeral 104, the silicon chip substrate will be cleaned. The silicon chip substrate coated with electrode is cut to suitable size. It is washed by the fresh water and acetone to remove the oil and grease particles on the substrate. Finally it is blown dry by the nitrogen gas. Put it in furnace under temperature 500□ to remove the moisture on the silicon chip substrate. Then the clean the silicon chip substrate is obtained.

As shown in FIG. 1, designating numeral 105, the spin coating process is used to prepare the anhydrous lead zirconium titanate film on the silicon chip substrate.

As shown in FIG. 1, designating numeral 106, the silicon chip substrate with spin coated anhydrous lead zirconium titanate film is heat treated at different temperatures. During the heat treatment, the organic impurity is removed in the first stage process, and it is called soft baking which includes the removal of solvent and the pyrolysis and oxidization of organic residues in the film. The solvent is usually removed at the ambient temperature to 250□. The second stage process is occurred between 300□ and 600□ (hard baking). In the range of this temperature, because of the combination formed by the condensation reaction, the anhydrous lead zirconium titanate film starts to become dense, and generates the crystal (the structure of lead zirconium titanate mine is started to form at 435□), so the sintering temperature should be higher than this temperature.

As shown in FIG. 1, designating numeral 107, the surface acoustic wave sensing element is produced. At first a layer of gold (conductive metal) electrode is coated on the anhydrous lead zirconium titanate piezoelectric film to become the Chemical Interaction Material (CIM) layer, such as the inter-digital transducer electrode, IDT electrode thereon.

As shown in FIG. 1, designating numeral 108, the surface of surface acoustic wave sensing element is modified and fixed by the streptavidin. The purpose is to fix the detecting antigen on the surface of surface acoustic wave sensing element, which is the golden chemical reaction material layer on the surface of anhydrous lead zirconium titanate film. The chemical bonding method or physical adsorption method can be used to combine the organism and the piezoelectric transducer to generate the bio-probe. Obviously, the sensing material is then connected to the surface of gold electrode. The phase change, frequency change caused by the surface perturbation and the property change of surface acoustic wave caused by the energy loss of surface acoustic wave are measured to understand the sensitivity and feasibility of the sensor.

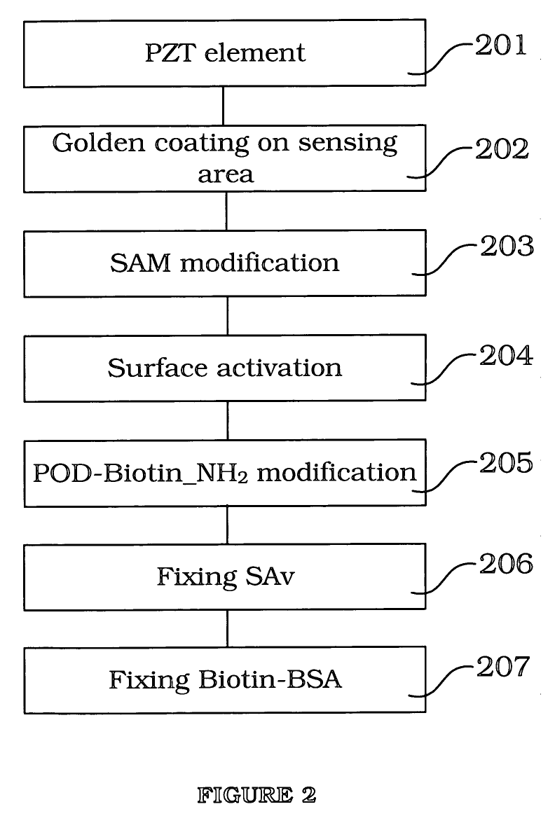

The more detail for fixing and modification on the surface of the streptavidin shown as FIG. 1, designating numeral 108 will be described as FIG. 2.

As FIG. 2, designating numeral 201 shows the produced PZT surface acoustic wave element.

Referring to FIG. 2, designating numeral 202, the golden is coated on the sensing area, the more detail can be shown as the previous FIG. 1, designating numeral 107.

As FIG. 2, designating numeral 203 illustrates modification on the surface of the sensing element using the self-assembled monolayer (SAM) modification method. The organism can be combined with the sensing element, wherein the sensing element can be soaked into the 16-mercaptopentadecanoic acid (16-MA) and then the 16-MA will become as the 16-MA film on the surface of the quartz crystal, such as the modification process.

In FIG. 2, designating numeral 204 shows that the surface activation will be carried out by using 1-Ethyl-3(3-Dimethylainopropyl) Carbodiimide Hydrochloride(EDC)and N-hydroxy Succinimide(NHS).

As FIG. 2, designating numeral 205 shows the 16-MA is reacted with Ethylenediamine and Perodate-Oxidized Dextran (POD), so that functional group such as formula COOH of the 16-MA will be activated to become as POD-Biotin-NH₂, such as the modification process.

Finally as FIG. 2, designating numeral 206 illustrates that using Perodate-Oxidized Dextran as the bridge is carried out, then the streptavidin (SAv) will be high affinity with the Biotin, so that the streptavidin can be fixed on Biotin-BSA as the sensing element.

In the embodiment, the thickness of anhydrous lead zirconium titanate film is up to 8 μm, and the width of metal line is only 2 μm.

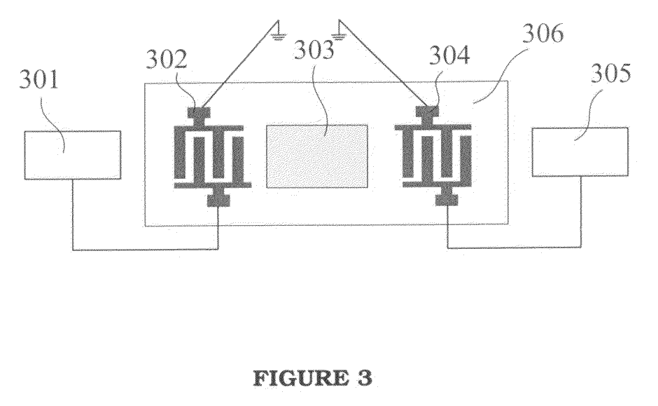

As FIG. 3, designating numeral 301 shows the signal generator, which can offer an external signal.

Still as FIG. 3, designating numeral 302 shows the input inter-digital transducer electrode as the sensor that can transfer the input signal to become as the surface acoustic wave.

As FIG. 3, designating numeral 303 illustrates the streptavidin film, which can be used as the bio-probe, which is called probe 303.

In FIG. 3, designating numeral 304 shows the output inter-digital transducer electrode that can transfer the surface acoustic wave that is generated by the input inter-digital transducer electrode, and is passed by the bio-probe, and is transferred to the output inter-digital transducer electrode, so that can be transferred back to as the electric wave signal.

As FIG. 3, designating numeral 305 shows the frequency counter, which can receive the output electric wave signal from the output inter-digital transducer electrode.

Finally, FIG. 3, designating numeral 306 shows the sensor substrate, which has the lead zirconium titanate layer with piezoelectric character, and it uses the piezoelectric character to carry on the surface acoustic wave detection.

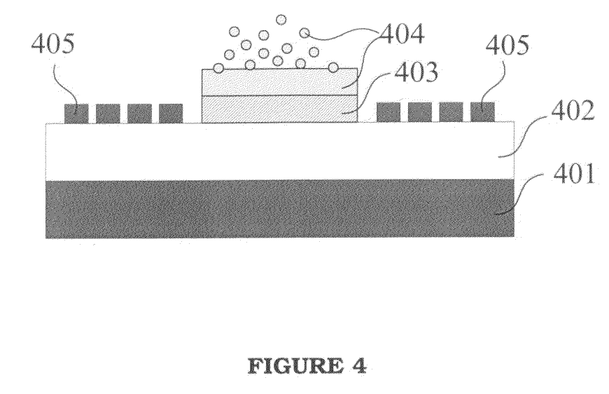

FIG. 4 shows a sectional view for the streptavidin surface acoustic wave sensing element of this invention.

As FIG. 4, designating numeral 401 is the silicon substrate.

Still as FIG. 4, designating numeral 402 is the lead zirconium titanate film.

FIG. 4, designating numeral 403 is the metal chemical reaction material layer, and the gold is usually used as the metal.

As FIG. 4, designating numeral 404 is the streptavidin film (the same as probe 303).

Finally FIG. 4, designating numeral 405 is the input/output inter-digital transducer electrode.

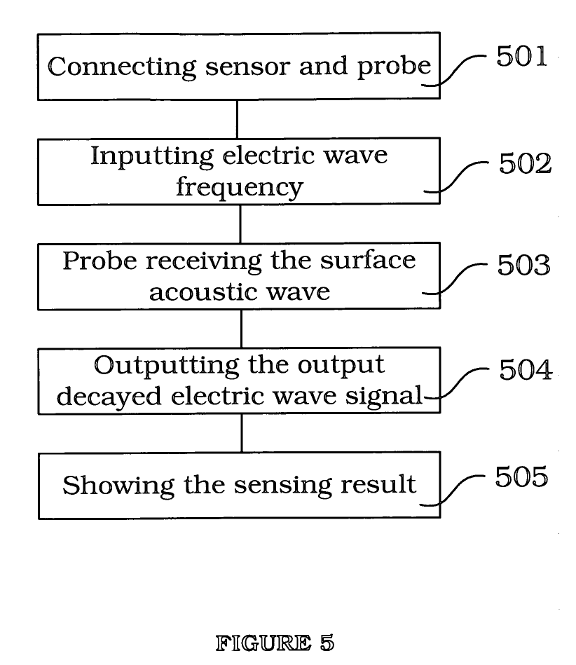

FIG. 5 (also referring to FIG. 3) can show the sensing method for the streptavidin surface acoustic wave sensing element of the invention. The principle of Sauerbrey equation is used in the embodiment of the invention. After verifying, the linear correlation will be formed between the amount of mass change on the surface of electrode and the amount of oscillating frequency of the crystal, and the detailed description is as follows:

$$Δ F = - \frac{( Δ M ) {F}^{2}}{( A ρ N )}$$

ΔF. The change of resonance frequency (Hz)
ΔM: The change amount of mass of unit area (g)
F: The original resonance frequency of the element (Hz)
(Aρ N): The property constant of the piezoelectric materials
A: The loading area (cm²)
ρ: Density ( The theory density of PZT: 7.5 g/ cm²)
N: Crystal frequency constant, N=F×t (F: Resonance frequency, t: thickness)

Firstly, as shown in FIG. 5, designating numeral 501, the sensor 302 and the probe 303 is connected.

Secondly, as shown in FIG. 5, designating numeral 502, the signal generator 301 can input the electric wave frequency, and then be transferred to become as the surface acoustic wave by using the input inter-digital transducer electrode 302.

And, as shown in FIG. 5, designating numeral 503, the probe 303 will receive the surface acoustic wave signal generated from the signal generator 301 and the input inter-digital transducer electrode 302.

Then, as shown in FIG. 5, designating numeral 504, the sound acoustic wave from the probe 303 will be transferred to become as the output decayed electric wave signal by using the output inter-digital transducer electrode 304.

Finally, as shown in FIG. 5, designating numeral 505, the frequency counter 305 can transfer the decayed electric wave signal from the output inter-digital transducer electrode 304 to display as the sensing result. Also, the result is calculated by using the frequency change, i.e. the principle of Sauerbrey equation.

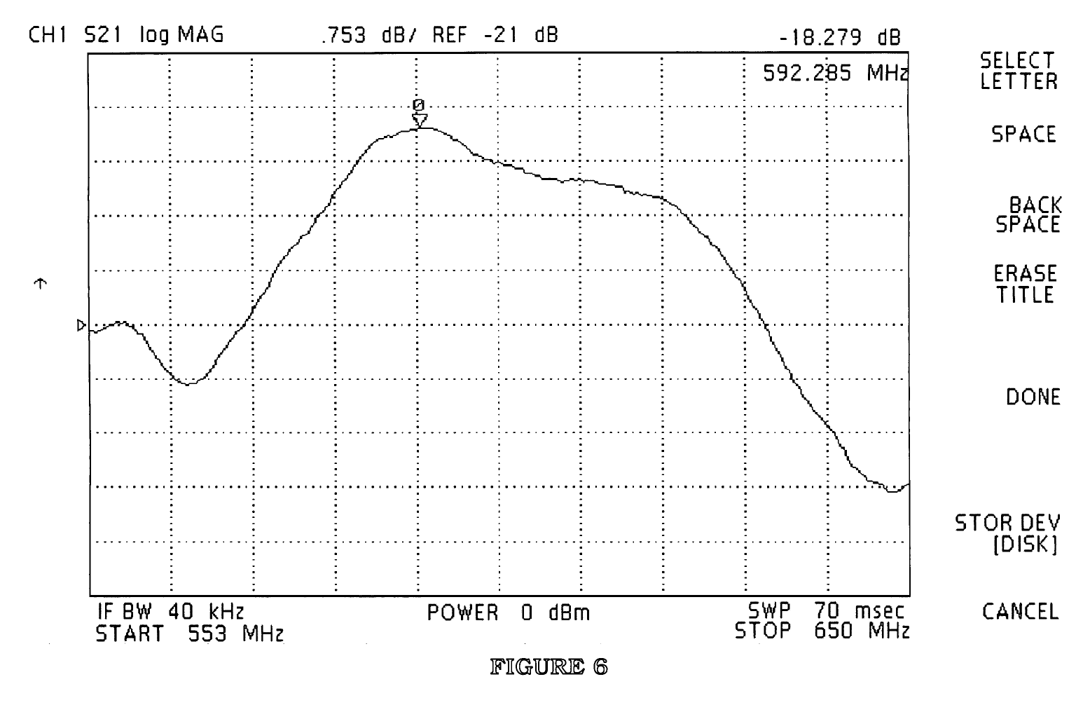

FIG. 6 shows the measurement of the central frequency. The frequency excited by the electrode is responded in 600 MHz. Therefore the response frequency will be measured about 600 MHz on the vector network analyzer. However, due to the theoretical phase velocity is at a single direction velocity, which is different from the vector direction velocity of the sound wave transferring in the practical. In addition, the energy loss of is occurred during the sound wave transferring process, due to the different character of the film, lifting error, thickness and roughness of the film. Therefore, the response peak signal will be obtained, the maximum central frequency is about 592.29 MHz, and the minimum loss of intervention is about 18.28 dB.

Thus, through the test of experiment, the sensitivity for the piezoelectric crystal of this invention can be raised to a level of 10⁻¹² g, so it is much suitable for the detecting of the biomolecular level. After the actual measurement in the embodiment, when the input resonant frequency is 600 MHz, the output resonant frequency outputted can reach 592 MHz, so the output loss of resonant frequency is extremely low.

According to the surface acoustic wave sensing principle, the invention uses the piezoelectric surface acoustic wave element of the anhydrous lead zirconium titanate as the surface acoustic wave sensing element, and combines the streptavidin to form the bio-probe, which becomes a new type immunosensor for the next generation.

It is understood that various other modifications will be apparent to and can be readily made by those skilled in the art without departing from the scope and spirit of this invention. Accordingly, it is not intended that the scope of the claims appended hereto be limited to the description as set forth herein, but rather that the claims be construed as encompassing all the features of patentable novelty that reside in the present invention, including all features that would be treated as equivalents thereof by those skilled in the art to which this invention pertains.

What is claimed is:

1. A method for manufacturing streptavidin surface acoustic wave immunosensor apparatus, comprising:
preparing a precursor solution of an anhydrous lead zirconium titanate (PZI) as an anhydrous lead zirconium titanate precursor,
forming the anhydrous lead zirconium titanate precursor to become as an anhydrous lead zirconium titanate powder;
ball milling the anhydrous lead zirconium titanate powder;
cleaning a silicon chip substrate;
forming an anhydrous lead zirconium titanate film on the silicon chip substrate;
heat treating the anhydrous lead zirconium titanate film and the silicon chip substrate;
forming a chemical interaction material layer on the anhydrous lead zirconium titanate film and an inter-digital transducer electrode thereon as a surface acoustic wave sensing element; and
modifying and fixing a streptavidin on a surface of the surface acoustic wave sensing element to form the streptavidin surface acoustic wave immunosensor apparatus.

2. The method according to claim 1, wherein forming the anhydrous lead zirconium titanate powder comprises the micro-powder-sol-gel method.

3. The method according to claim 1, wherein ball-milling the anhydrous lead zirconium titanate powder comprises the wet ball milling method.

4. The method according to claim 1, wherein forming the anhydrous lead zirconium titanate film comprises the spin coating method.

5. The method according to claim 1, wherein heat treating the anhydrous lead zirconium titanate film and the silicon chip substrate comprises a two-stage heat treating process.

6. The method according to claim 1, wherein forming a chemical interaction material layer on the anhydrous lead zirconium titanate film comprises a gold layer.

7. The method according to claim 1, wherein modifying and fixing a streptavidin on the surface of the surface acoustic wave sensing element comprises the chemical bonding method.

8. The method according to claim 1, wherein modifying and fixing a streptavidin on the surface of the surface acoustic wave sensing element comprises the physical adsorption.

9. A streptavidin surface acoustic wave immunosensor apparatus, comprising:
a signal generator offering an external signal;
an input inter-digital transducer electrode transferring an input signal to become as a surface acoustic wave;
a streptavidin film as a bio-probe;
an output inter-digital transducer electrode transferring the surface acoustic wave that being generated by the input inter-digital transducer electrode, the surface acoustic wave being passed by the bio-probe and being transferred to the output inter-digital transducer electrode, so that the surface acoustic wave being transferred back to become as an electric signal;
a frequency counter that receiving the output electric wave signal from the output inter-digital transducer electrode; and
a sensor substrate having a lead zirconium titanate layer to carry on the surface acoustic wave detection by using the piezoelectric character.

10. A measuring method for the streptavidin surface acoustic wave immunosensor, comprising:
connecting a sensor and a probe;
inputting an electric wave frequency by a signal generator, the electric wave frequency been transferred to become as the surface acoustic wave by using an input inter-digital transducer electrode;
receiving the surface acoustic wave signal generated from the signal generator and the input inter-digital transducer electrode by the probe;
transferring the sound acoustic wave from the probe to become as an output decayed electric wave signal by using the output inter-digital transducer electrode;
transferring a decayed electric wave signal from the output inter-digital transducer electrode by a frequency counter to display as the sensing result.

11. A measuring method of the streptavidin surface acoustic wave immunosensor apparatus using the frequency change, comprising:
connecting a sensor and a probe;
inputting an electric wave frequency by a signal generator, the electric wave frequency been transferred to become as the surface acoustic wave by using an input inter-digital transducer electrode;
receiving the surface acoustic wave signal generated from the signal generator and the input inter-digital transducer electrode by the probe;
transferring the sound acoustic wave from the probe to become as the output decayed electric wave signal by using the output inter-digital transducer electrode;
transferring a decayed electric wave signal from the output inter-digital transducer electrode by a frequency counter to display as the sensing result.

12. The method according to claim 11, wherein using the frequency change comprises the principle of Sauerbrey equation.
